(19) United States
(12) Patent Application Publication
(10) Pub. No.: US 2006/0257861 A1
(43) Pub. Date: Nov. 16, 2006

(54) Screening assay for inhibitors of severe acute respiratory syndrome (SARS) using SELDI-TOF Mass Spectrometry

(75) Inventors: Khalid M. Elased, Bellbrook, OH (US); Mariana Morris, Yellow Springs, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: Wright State University, Dayton, OH (US)

(21) Appl. No.: 11/433,050

(22) Filed: May 12, 2006

Related U.S. Application Data
(60) Provisional application No. 60/680,191, filed on May 12, 2005.

(51) Int. Cl. C12Q 1/70

(52) U.S. Cl. 435/5

(57) ABSTRACT

Mass spectrometric methods directed to screening libraries of compounds for agents that inhibit entry of Severe Acute Respiratory Syndrome (SARS) coronavirus (CoV) into cells are provided, along with methods for comparatively evaluating inhibitors of the SARS CoV.

### CROSS REFERENCE TO RELATED APPLICATIONS

This application claims the benefit of U.S. Provisional Application No. 60/680,191 filed May 12, 2005.

### GOVERNMENT INTERESTS

The present invention was federally funded, in part, by the NHLBI—grant number 5R01HL069319-03(S).

### BACKGROUND OF THE INVENTION

The present invention relates generally to the science and technologies of bio-processing. Specifically, the present invention is directed to a quantitative assay method using Surface-Enhanced Laser Desorption/Ionization-Top of Flight (“SELDI-TOF”) Mass Spectrometry to screen libraries of agents for inhibition of Angiotensin Converting Enzyme 2 (ACE2) and the SARS coronavirus.

SELDI-TOF Mass Spectrometry is an increasingly popular bio-analytical method due in part to the development of techniques that avoid protein fragmentation during the process of volatization (e.g. desorption) and ionization. Other advantages of using SELDI-TOF Mass Spectrometry comprise the ability to identify proteins in a complex sample mixture with high resolution. The use of Mass Spectrometry for protein analysis is described in U.S. Pat No. 5,118,937 to Hillenkamp et al., U.S. Pat. No. 5,617,060 to Hutchens et al., and WO 98/59360 to Hutchens and Yip. The present invention describes the functional use of SELDI-TOF Mass Spectrometry to accurately quantify enzymatic activity of ACE2, the enzymatic protein known to provide the vehicle for entry of the SARS coronavirus into cell, and to evaluate effectors (i.e. activators or inhibitors) of ACE2. The present invention uniquely describes methods of using SELDI-TOF Mass Spectrometry to screen for pharmacological inhibitors of Severe acute respiratory syndrome (SARS). The invention also provides a high throughput screening assay to identify pharmacological agents from libraries of SARS inhibitors.

The susceptibility of cells to viral infection is determined by their ability to support virus entry, replication, maturation and egress. Virus entry depends on the expression of specific cellular receptors. A wide range of carbohydrates, protein and lipids can serve as virus receptors. Identification of virus receptors is crucial for understanding the pathogenesis of viral diseases.

In 2003, a novel coronavirus was identified as the etiological agent for SARS, which had recently emerged as a serious disease threat in Southeast Asia. Angiotensin converting enzyme 2 (ACE2) was shortly thereafter determined to be a functional receptor for the SARS coronavirus. While much investigation has taken place regarding the molecular biochemistry of the ACE2 protein and SARS coronavirus interaction, and some researchers have suggested pharmacological mechanisms involving, for example, ACE2 antibodies, there is a clear need in the art for effective inhibitors of the SARS coronaviral activity, and methods for rapidly screening libraries of compounds for such effectors.

### SUMMARY OF THE INVENTION

Accordingly, the present invention is directed to a bio-analytical method that comprises detecting specific products of an enzymatic reaction for which the substrate is known in minute quantities of crude biological samples deposited on a protein chip (Ciphergen Biosystems, CA), which is thereafter subjected to Surface Enhanced Laser Desorption Ionization/Time of Flight (SELDI-TOF) Mass Spectrometry, analyzed and quantified for products of the enzymatic reaction.

The present invention provides methods for quantitatively measuring the physiologically relevant enzymatic activity ACE2 under certain conditions, in the presence of effecting agents. Advantages of the present invention comprise the need only for minute quantity of sample to perform the assay, the ability to scale up the assay, and to process a large number of samples in a very short amount of time and at a low cost.

In one aspect of the present invention, a method of identifying and quantifying products of the renin-angiotensin system in test samples comprising potential effectors of ACE2. The precursor enzyme, Angiotensin I or Angiotensin II, is coated onto a protein chip, for example, a weak cation exchange proteinChip® (WCX2). Test samples comprising prospective agents mixed with purified ACE2 are prepared and spotted onto the protein chip. Thereafter the loaded chip may be washed to remove unbound proteins, salts and other contaminants, and is read using a SELDI-TOF mass spectrometer. A spectral analysis is performed by using a commercially available software (version 3.1 Ciphergen Biosystems), and finally the enzymatic products, Ang1-9 or Ang1-7, respectively, are identified and quantified using peak height intensity and area under the curve calculations.

One embodiment of the present invention provides a mass spectrometric method of screening for agents that inhibit entry of Severe Acute Respiratory Syndrome (SARS) coronavirus (CoV) into cells. The method comprises: a. preparing a plurality of test agent samples and at least one test control sample; b. adding purified Angiotensin Converting Enzyme 2 (ACE2) to the control sample, and to each test agent sample, to form a plurality of sample/ACE2 mixtures; c. pre-coating a protein chip with Angiotensin II, or Angiotensin I, depending on the enzymatic product being measured; d. applying a quantity of each sample/ACE2 mixture to the pre-coated protein chip; e. permitting an enzyme reaction to proceed for a sufficient amount of time; f. subjecting the chip to a mass spectrometric technique; g. analyzing the protein chip both qualitatively and quantitatively for Ang1-7 (if the precursor is Angiotensin II), or Ang1-9 (if the precursor is Angiotensin I); wherein a quantitative decrease in Ang1-7 relative to the control indicates that a test agent is an inhibitor of ACE2, and further wherein an inhibitor of ACE2 by this method comprises an inhibitor of SARS CoV. A further embodiment provides that the method comprises a high throughput method.

Another embodiment of the present invention provides a method of comparatively evaluating inhibitors of Severe Acute Respiratory Syndrome (SARS) coronavirus (CoV). The method involves making a relative comparison of the inhibition efficacy of ACE2 inhibitors, where the quantity of enzymatic product correlates inversely to the degree of inhibition, and where efficacy of inhibition of ACE2 correlates directly to efficacy of inhibition of SARS CoV. The method comprises: a. preparing a plurality of inhibitor samples; b. incubating the inhibitor samples with purified ACE2, resulting in a plurality of effector sample/ACE2 mixtures; c. coating a protein chip with Angiotensin I; d. loading the plurality of mixtures onto the coated protein chip; e. subjecting the loaded chip to SELDI-TOF mass spectrometry; f. generating spectral data and conducting a quantitative analysis of the spectral data to determine a level of Ang1-7 for each sample/ACE2 mixture; and g. comparing the determined level of Ang1-7 for each inhibitor, and ranking them relative to one another for inhibition efficacy, wherein the amount of Ang1-7 is inversely proportional to efficacy of inhibition.

These and other features of the present invention will be more fully understood from the following detailed description of the invention taken together with the accompanying drawings. It should be noted that the scope of the invention is defined by the claims and should not be construed as limited by the figures or by the specific discussion of features and advantages set forth in the description.

### BRIEF DESCRIPTION OF THE FIGURES

FIG. 1: SELDI-TOF-MS assay for ACE2 activity in kidney extract obtained from wild (WT) and ACE2 knockout (KO) mice. AngII is used as a substrate for ACE2. Note the diminishment of Ang1-7 (m/z, 899) in the KO mice.

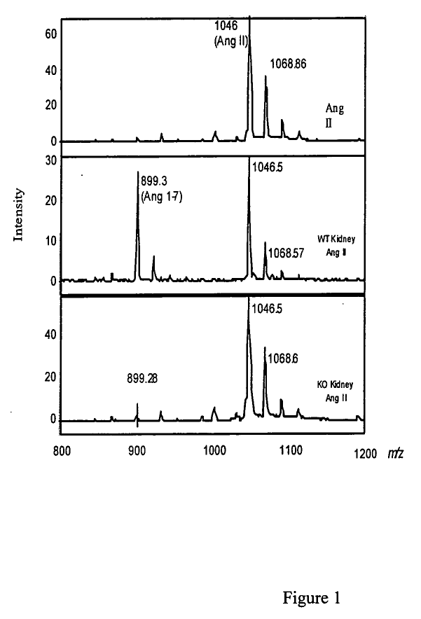

FIG. 2: ACE1 activity in the plasma. ACE1 activity is detected by the formation of the peak corresponding to m/z 1046 (AngII). MLN 4760 is a selective inhibitor of ACE2. There was no effect of MLN 4760 on the formation of AngII.

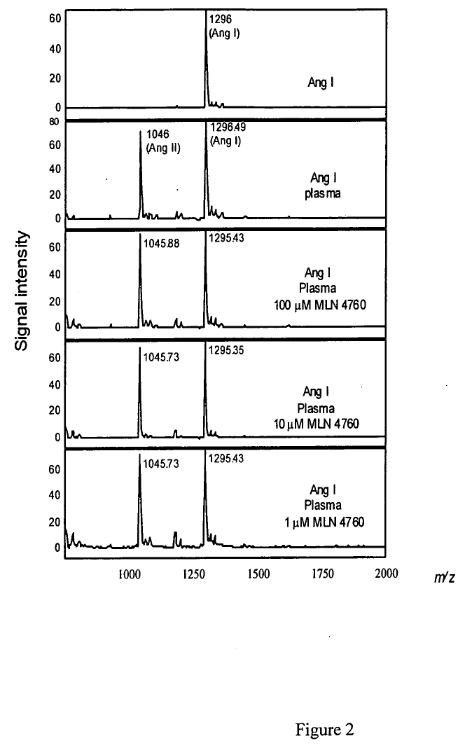

FIG. 3: SELDI-TOF-MS ACE assay. Kidney extract (source of ACE1 & ACE2) were incubated with AngI A: kidney extract control; B: AngI; C: AngI+kidney extract, showing peptides generated from ACE1 & ACE2; D: AngI+kidney extract+MLN 4760 (ACE2 inhibitor). In D, AngII is increased C since MLN 4760 inhibited the cleavage of AngI to Ang (1-7) without affecting ACE1.

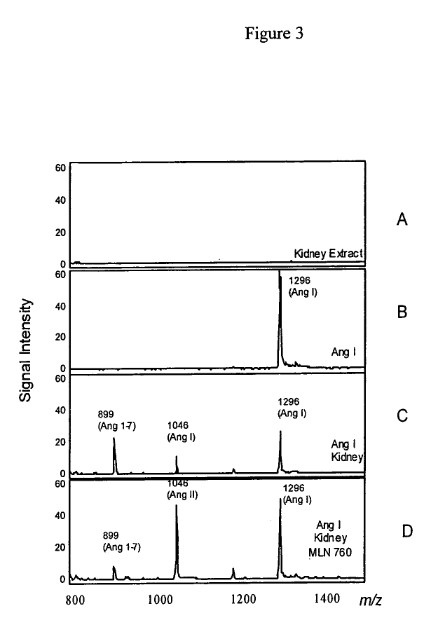

### DETAILED DESCRIPTION OF THE PREFERRED EMBODIMENT

The standard method for the identification and quantitative analysis of proteins uses two dimensional Poly Acrylamide Gel Electrophoresis (PAGE) followed by mass spectrometry. Although this method provides excellent resolution, it is time consuming, labor intensive and requires large amounts of sample, which hampers fast analysis of a large number of samples. Furthermore, multiple manipulations of proteins increases the risk for permanent denaturation, partial or total degradation, which are likely to negatively impact their function, for example catalytic in the case of enzymes.

Surface Enhanced Laser Desorption Ionization-Time of Flight (SELDI-TOF) Mass Spectroscopy is a fairly recently developed technology for protein analysis that combines two powerful analytical techniques: chromatography and mass spectrometry. Proteins are retained on a ProteinChip® array which consists in a solid-phase chromatographic surface that has been modified in a manner such that it retains proteins based on their physico-chemical or biological properties.

Biochemical chromatographic arrays are particularly well-suited to investigate specific molecular recognition mechanisms such as antibody-antigen or receptor-ligand interactions. Among other advantages, this method only requires minute sample volumes, in the micro liter order of magnitude, is compatible with spotting crude biological samples, and preserves the native structures of the proteins to be analyzed.

After washing the arrays to eliminate excess of unbound molecules, salts or contaminants, and drying, a solution containing an Energy Absorbing Molecule is added to the ProteinChip® which is thereafter inserted into the ProteinChip® reader to measure the molecular weights of the bound proteins. The ProteinChip® reader is a Laser Desorption/Ionization Time of Flight mass spectrometer. A UV nitrogen laser source irradiates the molecules present in the samples, which causes their desorption/ionization as gaseous ions whose mass-to-charge ratio (m/z) is measured based on the velocity of each molecular ion through an ion chamber. An analog-to-digital converter linked to a personal computer processes the signals, and each detected protein shows as a peak on a spectrum. The height of the peaks and area-under-the curve permit quantification of each protein species detected in the sample. The quantities of materials required and the speed and accuracy of the technique make it well-suited for adaptation to high through put screening assays.

Indeed, the versatility of the SELDI-TOF Mass spectrometry technique has led to a broad range of applications in clinical diagnostic and proteomics. However, this technique had not been used to measure disease-associated modification of enzymatic activity associated with the Renin-Angiotensin System (RAS) prior to work by the present inventors, disclosed in U.S. applicant Ser. No. 11/292,806, incorporated fully herein by this reference. In particular, application of this method to screen for agents which inhibit the SARS coronavirus are heretofore unknown.

Enzymes are molecules that reversibly and specifically bind to a variety of substrates to enable chemical reactions that otherwise are impossible or difficult to achieve under normal physiological conditions to occur. Because enzymes are proteins, their structure is very sensitive to a variety of environmental factors that influence the kinetics of the enzymatic equilibrium, and the enzymatic function. The affinity between enzyme, substrate and/or cofactor, which largely depends on the conformational fit between these reagents, drives the enzymatic kinetics of the reaction. It is indeed very well-known in the art that modifying the conformational fit between an enzyme and its ligand by changing the structure of either the enzyme or its substrate, for example by attaching a chromophore, changing the environment of the enzymatic reaction, or by genetically engineering the enzyme, may substantially modify the kinetics of the enzymatic reaction. Current experimental methods used to analyze proteolytic enzyme activity employ specially designed chromogenic substrates to enable measurement of activity by spectrophotometry. However, due to the use of a chromogenic substrate, the kinetics of the enzymatic reaction may not parallel that of the enzymatic reaction with natural substrates. Therefore, there may be a substantial discrepancy between the experimental and in vivo enzymatic activities

Enzymes play a central role in the general metabolism of all living beings, and have therefore a vital physiological function. They often operate in concert thereby realizing enzymatic systems such as the renin-angiotensin system, or RAS, which plays a crucial role in the regulation of blood pressure, cardiac function, and electrolyte balance. Angiotensin converting enzymes (ACE) are central actors of the Renin-Angiotensin System; they participate to the production of Angiotensin I (inactive) and II (vasoconstrictor). In addition to the circulating Renin-Angiotensin System, there is also tissue expression of this system in the brain, kidney, pancreas, and other organs. Enzymes of the Renin-Angiotensin System raise substantial clinical interest as biomarkers for pathological conditions such as hypertension, diabetes, or renal dysfunction.

ACE2 is a homolog of ACE1, both of which are central enzymes in the renin-angiotensin-system (RAS) involved in blood pressure regulation, as well as in fluid and salt balance. Whereas ACE1 cleaves the decapeptide Angiotensin I (AngI) into an octapeptide Angiotensin II (AngII), ACE2 functions as a carboxypeptidase, cleaving a single residue from AngI, generating Ang1-9, and a single residue from AngII to generate Ang1-7. An increase in ACE 1 activity results in an increase in AngII, measured by SELDI-TOF Mass Spectrometry, and indicate a high risk in the onset of hypertension. Targeted disruption of murine ACE2 results in increased AngII levels, impaired cardiac contractility in older mice, and upregulation of hypoxia-induced genes in the heart. Loss of ACE1 on an ACE2 background reverses this heart phenotype. Thus, ACE2 is a negative regulator of the RAS and counterbalances the function of ACE (Crackower et al. Nature).

The susceptibility of cells to viral infection is determined by their ability to support virus entry, replication, maturation and egress. Virus entry depends on the expression of specific cellular receptors. A wide range of carbohydrates, protein and lipids can serve as virus receptors. Identification of virus receptors is crucial for understanding the pathogenesis of viral diseases.

In 2003, a novel coronavirus was identified as the etiological agent for SARS, which had recently emerged as a serious disease threat in Southeast Asia. The SARS pathogen triggers atypical pneumonia characterized by high fever and severe dyspnea. The death rate following infection approached˜10% due to the development of acute severe lung failure. The high lethality of SARS infections, their enormous economic and social impact, fears of renewed outbreaks of SARS as well as the feared misuse of such viruses as biological weapons make it paramount to understand the disease pathogenesis of SARS and acute severe lung failure.

Angiotensin converting enzyme 2 (ACE2) was discovered to be a functional receptor for the SARS coronavirus. Hence, the present inventors developed screening assays for agents which inhibit SARS by utilizing the knowledge of this relationship between ACE2 and SARS. Inhibition may be ascertained by fixing an ACE2 substrate, AngII, on the ProteinChip, mixing potential inhibitory agents with ACE2, exposing the mixture to the ProteinChip, and measuring the impact on the formation of the enzymatic products in comparison to their formation in the absence of the potentially inhibitory agent. This is illustrated in Example 1, below.

One embodiment of the present invention is directed to a method for screening for SARS coronavirus inhibitors by screening for ACE2 inhibitors. An ACE2 enzymatic product precursor, either AngI or AngII, is coated onto a protein chip, while ACE2 is incubated with test agents. The test agent/ACE2 mixture is loaded onto the coated chip, and the loaded chip is permitted to incubate sufficiently for the enzymatic reaction to occur under control conditions, and is then subjected to SELDI-TOF mass spectrometry. Identification of the degradation of AngII to Ang1-7 is used to measure the activity of the inhibitors. These inhibitors will compete with the SARS coronavirus to bind to the ACE2 (receptor).

Specifically, a mass spectrometric method of screening for agents that inhibit entry of Severe Acute Respiratory Syndrome (SARS) coronavirus (CoV) into cells is provided. The method comprises: a. preparing a plurality of test agent samples and at least one test control sample; b. adding purified Angiotensin Converting Enzyme 2 (ACE2) to the control sample, and to each test agent sample, to form a plurality of sample/ACE2 mixtures; c. pre-coating a protein chip with Angiotensin II, or Angiotensin I, depending on the enzymatic product being measured; d. applying a quantity of each sample/ACE2 mixture to the pre-coated protein chip; e. permitting an enzyme reaction to proceed for a sufficient amount of time; f. subjecting the chip to a mass spectrometric technique; g. analyzing the protein chip both qualitatively and quantitatively for Ang1-7 (if the precursor is Angiotensin II), or Ang1-9 (if the precursor is Angiotensin I); wherein a quantitative decrease in Ang1-7 relative to the control indicates that a test agent is an inhibitor of ACE2, and further wherein an inhibitor of ACE2 by this method comprises an inhibitor of SARS CoV. A further embodiment provides that the method comprises a high throughput method.

According to a more specific embodiment, the mass spectrometric technique comprises SELDI-TOF mass spectrometry. This technique is particularly suitable for high throughput screening assays, and one specific embodiment provides a high throughput screening assay partially automated by robotics.

Another specific embodiment is directed to the inventive method wherein analyzing the chip quantitatively comprises (i) generating spectral data using commercially available software compatible for this purpose; (ii) analyzing the spectral data to identify Ang1-7; and (iii) subjecting the spectral data to an algorithm that permits quantification of the Ang1-7. In certain embodiments where the protein chip is pre-coated with Angiotensin II, the protein chip is analyzed for the presence and quantity of Ang1-7, while in other embodiments the protein chip is pre-coated with Angiotensin I and the protein chip is analyzed for the presence and quantity of Ang1-9.

Any algorithm capable of generating quantifying information may be applied to the data to obtain quantification of the enzymatic product. In particular embodiments, quantification of the Ang1-7 is by obtaining a measurement of the peak intensity or Area Under the Curve (AUC) of Ang1-7 and expressing the measurement as a percentage of the peak intensity or AUC of the Angiotensin II. In other particular embodiments, quantification of Ang1-9 is by obtaining a measurement of the peak intensity or AUC of Ang1-9 and expressing the measurement as a percentage of the peak intensity or AUC of the Angiotensin I.

Another embodiment of the present invention is directed to a method of comparatively evaluating inhibitors of Severe Acute Respiratory Syndrome (SARS) coronavirus (CoV). The method involves making a relative comparison of the inhibition efficacy of ACE2 inhibitors, where the quantity of enzymatic product correlates inversely to the degree of inhibition, and where efficacy of inhibition of ACE2 correlates directly to efficacy of inhibition of SARS CoV. The method comprises: a. preparing a plurality of inhibitor samples; b. incubating the inhibitor samples with purified ACE2, resulting in a plurality of effector sample/ACE2 mixtures; c. coating a protein chip with Angiotensin I; d. loading the plurality of mixtures onto the coated protein chip; e. subjecting the loaded chip to SELDI-TOF mass spectrometry; f. generating spectral data and conducting a quantitative analysis of the spectral data to determine a level of Ang1-7 for each sample/ACE2 mixture; and g. comparing the determined level of Ang1-7 for each inhibitor, and ranking them relative to one another for inhibition efficacy, wherein the amount of Ang1-7 is inversely proportional to efficacy of inhibition.

The following example is intended to be illustrative of one embodiment of the present invention and should not be construed to limit the scope thereof.

### EXAMPLE 1

This example illustrates the use of SELDI-TOF MS technology in a high throughput screening assay designed to screen libraries of agents for SARS CoV inhibitors.

ProteinChips are pre-coated with the precursor enzyme, AngII. The Protein Chip may simultaneously screen up to 16 test-agents. The test-agent samples are mixed with purified ACE2 enzyme. One ml of each test-agent/ACE2 mixture is applied to the pre-coated ProteinChip. The enzyme reaction is stopped by application of matrix CHCA after 15-30 minutes. Protein chips are analyzed using the ProteinChip® reader. Activity of ACE2 is determined by measuring the peak intensity or Area Under the Curve (AUC) of the product, Ang 1-7 (899 m/z) and expressing it as a percentage of the peak intensity and AUC of the precursor AngII. Inhibitors of ACE2 by this mechanism will also inhibit SARS and demonstrate blocking or diminishment of the formation of Ang1-7.

What is claimed:

1. A mass spectrometric method for screening of agents that inhibit entry of Severe Acute Respiratory Syndrome (SARS) coronavirus (CoV) into cells, the method comprising:
a. preparing a plurality of test agent samples and at least one test control sample;
b. adding purified Angiotensin Converting Enzyme 2 (ACE2) to the control sample, and to each test agent sample, to form a plurality of sample/ACE2 mixtures;
c. pre-coating a protein chip with Angiotensin II;
d. applying a quantity of each sample/ACE2 mixture to the pre-coated protein chip;
e. permitting an enzyme reaction to proceed for a sufficient amount of time;
f. subjecting the chip to a mass spectrometric technique;
g. analyzing the protein chip both qualitatively and quantitatively for Ang1-7;
wherein a quantitative decrease in Ang1-7 relative to the control indicates that a test agent is an inhibitor of ACE2, and further wherein an inhibitor of ACE2 by this method comprises an inhibitor of SARS CoV.

2. The method according to claim 1, wherein the mass spectrometric technique comprises SELDI-TOF mass spectrometry.

3. The method according to claim 1 wherein the method comprises a high throughput screening assay and is performed in part by robotics.

4. The method according to claim 1, wherein analyzing the chip quantitatively comprises (i) generating spectral data using commercially available software compatible for this purpose; (ii) analyzing the spectral data to identify Ang1-7; and (iii) subjecting the spectral data to an algorithm that permits quantification of the Ang1-7.

5. The method according to claim 1, wherein step “c” is replaced with: pre-coating the protein chip with Angiotensin I, and step “g” is replaced with: analyzing the protein chip both qualitatively and quantitatively for Ang1-9.

6. The method according to claim 1, wherein quantification of the Ang1-7 is by obtaining a measurement of the peak intensity or Area Under the Curve (AUC) of Ang1-7 and expressing the measurement as a percentage of the peak intensity or AUC of the Angiotensin II.

7. The method according to claim 5, wherein quantification of the Ang1-9 is by obtaining a measurement of the peak intensity or AUC of Ang1-9 and expressing the measurement as a percentage of the peak intensity or AUC of the Angiotensin I.

8. A method of comparatively evaluating inhibitors of Severe Acute Respiratory Syndrome (SARS) coronavirus (CoV) by comparing inhibitors of ACE2, the method comprising:
a) preparing a plurality of inhibitor samples;
b) incubating the inhibitor samples with purified ACE2, resulting in a plurality of effector sample/ACE2 mixtures;
c) coating a protein chip with Angiotensin I;
d) loading the plurality of mixtures onto the coated protein chip;
e) subjecting the loaded chip to SELDI-TOF mass spectrometry;
f) generating spectral data and conducting a quantitative analysis of the spectral data to determine a level of Ang1-7 for each sample/ACE2 mixture;
g) comparing the determined level of Ang1-7 for each inhibitor, and ranking them relative to one another for inhibition efficacy, wherein the amount of Ang1-7 is inversely proportional to efficacy of inhibition.

9. The method according to claim 8, wherein step “c” is replaced with: pre-coating the protein chip with Angiotensin II, step “f” is replaced with: generating spectral data and conducting a quantitative analysis of the spectral data to determine a level of Ang1-7, and step “g” is replaced with comparing the determined level of Ang1-9 for each inhibitor, and ranking them relative to one another for inhibition efficacy.
